(19) United States
(12) Patent Application Publication
(10) Pub. No.: US 2003/0229272 A1
(43) Pub. Date: Dec. 11, 2003

(54) Universal scissors joint apparatus

(76) Inventors: Anthony J. Mulac, East Jordan, MI (US); Daniel K. Farley, Traverse City, MI (US)

Correspondence Address: MCANDREWS HELD & MALLOY, LTD, 500 WEST MADISON STREET, SUITE 3400, CHICAGO, IL, 60661

(21) Appl. No.: 10/164,487

(22) Filed: Jun. 6, 2002

(51) Int. Cl. A61B 1/32

(52) U.S. Cl. 600/234

(57) ABSTRACT

A universal joint apparatus comprises clamps, a locking mechanism, and a rod connecting the clamps and the locking mechanism. At least one clamp is a scissors clamp, i.e. a clamp comprising two segments fastened by a pivot. The scissors clamp generates extra compressive force on the object being held, providing a stable and rigid universal joint. Clamps are able to rotate with respect to each other, allowing for greater flexibility in usage. The universal joint apparatus is capable of being added to a support frame between other components

### BACKGROUND OF THE INVENTION

The present invention relates to surgical apparatus for retracting a patient's anatomy during an operation to provide exposure of the operative site. More particularly, the present invention relates to a universal scissors joint apparatus that is sturdy, stable, readily adjustable, and easily sterilized.

Surgical operations often require prolonged access to the internal anatomy of a patient. Retractors are used to hold back tissue around the surgical site, granting the surgeon the needed access. While hand-held retractors may be used during surgeries, it is often desirable to use mechanically mounted retractors.

Mechanical retractors are typically mounted to some kind of support structure. This support structure often takes the form of a frame surrounding part or all of the operating table. The frame may contain rails to which clamps may be attached. These clamps may connect the frame directly to a retractor, or to accessory rails to which retractors or additional rails may be connected. Greater flexibility in universal joint clamps alleviates some of the deficiencies of previous rail clamps in comparison to the manual application of retractors.

Universal joints must be sterilized before being brought into the operating area. Many previous universal joints have separable components which require more care and effort for sterilization due to the need to disassemble and reassemble the components. Universal joints with unitary designs permit sterilization without the need to disassemble the joints.

Some previous universal joints have used threaded locking mechanisms, which require lubrication and maintenance. Cam locking mechanisms require less maintenance and provide a much easier and more effective system for locking and unlocking the clamps.

Because other components may be secured to the frame, it is desirable for a universal joint to have the capability of being added to the frame between secured components.

While universal joints with the above features have been designed, it is desirable to have a universal joint with even greater ease of use, flexibility, stability, and rigidity.

### BRIEF SUMMARY OF THE INVENTION

The present invention provides a universal joint apparatus. The present invention comprises clamps, a locking mechanism, and a rod associating the clamps with the locking mechanism. At least one clamp is a scissors clamp, i.e. a clamp comprising a first segment and a second segment, with the segments fastened by a pivot. The scissors clamp generates extra compressive force on the object being held, providing a stable and rigid universal joint. Clamps are able to rotate with respect to each other, allowing for greater flexibility in usage. The present invention is capable of being added to a support frame between other components.

These and other features, aspects, and advantages of the present invention will be better understood with reference to the accompanying drawings, descriptions, and claims.

### BRIEF DESCRIPTION OF SEVERAL VIEWS OF THE DRAWINGS

FIG. 1 is a top perspective view of an embodiment of the present invention.

FIG. 2 is a partial bottom perspective view of the present invention.

FIG. 3 is a side view, in partial cross-section of an embodiment of the present invention.

FIG. 4 is a top view, in partial cross-section of an embodiment of a cam locking mechanism of the present invention.

FIG. 5 is a side view, in partial cross-section of an embodiment of a cam locking mechanism and rod of the present invention.

FIG. 6 is a top view, in partial cross-section, illustrating the operation of locking and unlocking an embodiment of the present invention.

The foregoing summary, as well as the following detailed description of the preferred embodiments of the present invention, will be better understood when read in conjunction with the appended drawings. For the purpose of illustrating the preferred embodiments of the present invention, there is shown in the drawings, embodiments which are presently preferred. It should be understood, however, that the present invention is not limited to the arrangements and instrumentality shown in the attached drawings.

### DETAILED DESCRIPTION OF THE INVENTION

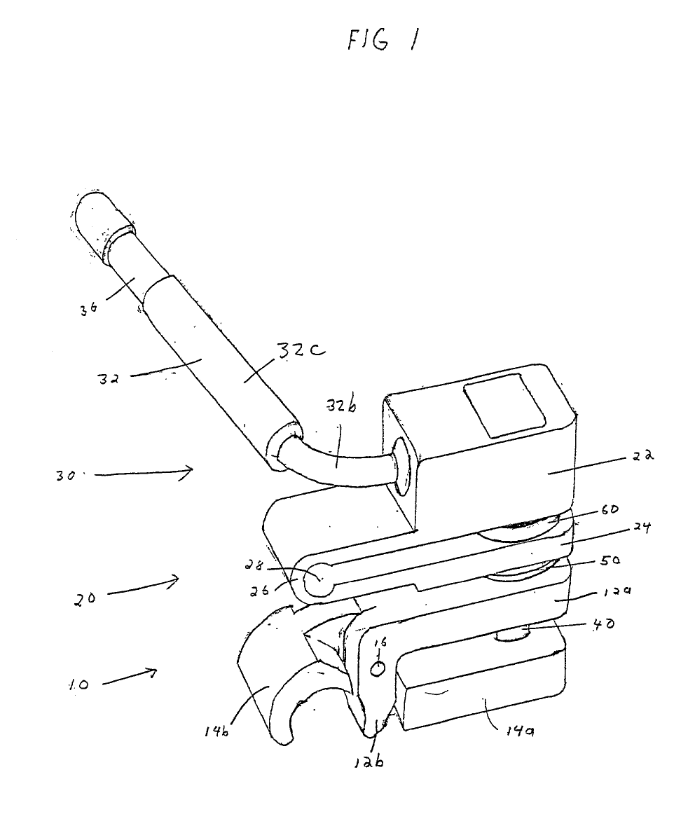

Referring to FIG. 1, the disclosed embodiment of the universal scissors joint apparatus includes a first clamping member referred to as a scissors clamp 10, a second clamping member referred to as a circle clamp 20, a cam locking mechanism 30, and a rod 40. The rod 40 associates the cam locking mechanism 30, the circle clamp 20, and the scissors clamp 10.

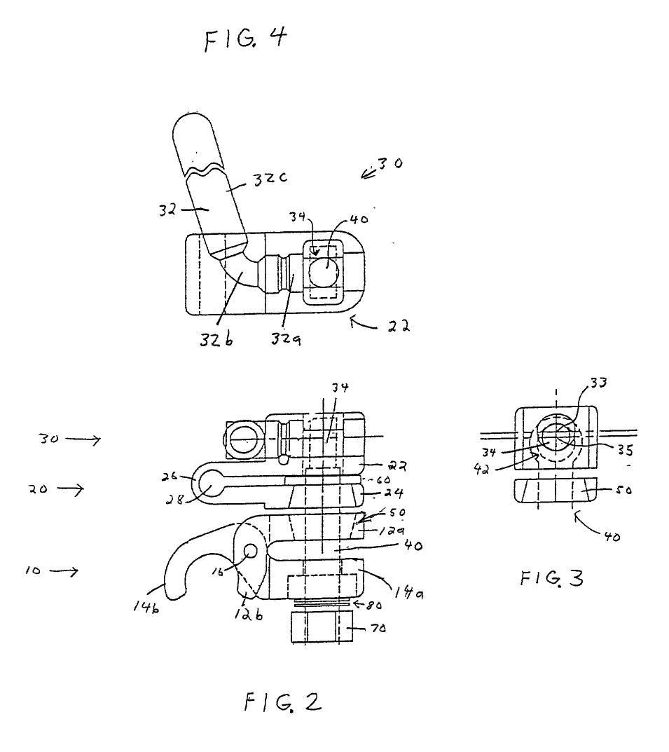

Referring to FIGS. 1, 2, and 3, the scissors clamp 10 includes two segments connected at a pivot 16, similar to a scissors, so that the two segments cross each other at the pivot 16. The first segment 12 includes an upper portion, referred to as an upper handle 12a, of the scissors clamp 10 proximal of the pivot 16 and engaging the rod 40; the first segment 12 further includes two lower portions, referred to as lower grippers 12b, of the scissors clamp 10 distal of the pivot 16. The second segment 14 includes a lower portion, referred to as a lower handle 14a, of the scissors clamp 10 proximal of the pivot 16; the second segment 14 further includes an upper portion, referred to as an upper gripper 14b, of the scissors clamp 10 distal of the pivot 16. The grippers 12b, 14b of the scissors clamp 10 are shaped so as to contour the surface of the object (not shown) to which the clamp is being attached. The inner surface of the upper gripper 14b of the scissors clamp 10 may include indentations 14c. These indentations 14c may be located opposite the lower grippers 12b. The handles 12a, 14a of the scissors clamp 10 are separated by a gap that allows the scissors clamp 10 to be squeezed, creating a tighter grip on the instrument being held by the grippers 12b, 14b of the clamp. The handles 12a, 14a of the scissors clamp 10 each have an opening that allows the rod 40 to pass through. A bushing 50 may be used. The bushing 50 may surround the rod 40 and fit into the opening in the upper handle 12a.

The circle clamp 20 includes an upper portion 22 and a lower portion 24 connected to form a single piece. The upper portion 22 and lower portion 24 are connected at a circular shaped fulcrum 26. The fulcrum 26 has a circular hole 28 in it. The hole 28 allows for the insertion of a retractor, rail, or other object (not shown). Except for the connection at the fulcrum 26, a gap exists between the upper portion 22 and lower portion 24 of the circle clamp 20. The gap allows the circle clamp 20 to be squeezed, tightening the grip on the object being held in the circle clamp 20. A spacer 60 may lie within this gap. Both the upper portion 22 and lower portion 24 of the circle clamp 20 have an opening through which the rod 40 may pass. The opening in the lower portion 24 may fit the same bushing 50 that engages the scissors clamp 10.

Referring to FIGS. 1, 4, and 5, the locking mechanism 30 includes a handle 32 connected to a cam 34. The handle 32 consists of a first straight portion 32a, an elbow 32b, and a second straight portion 32c. The first straight portion 32a projects straight out from the cam 34, then the elbow 32b curves at an angle before the second straight portion 32c projects straight out from the elbow 32b. The second straight portion 32c of the handle 32 includes a recessed area 36. The cam 34 may be shaped asymmetrically with respect to the center axis 33 of the handle, so that the cam's center axis 35 is not aligned with the handle's center axis 33. The cam 34 is positioned through an eyehole 42 in the rod 40. Alternatively, the cam's center axis 35 may be aligned with the handle's center axis 33 where the cam 34 is not circular but instead has different radial lengths along different points of its perimeter, as will be appreciated by those skilled in the art.

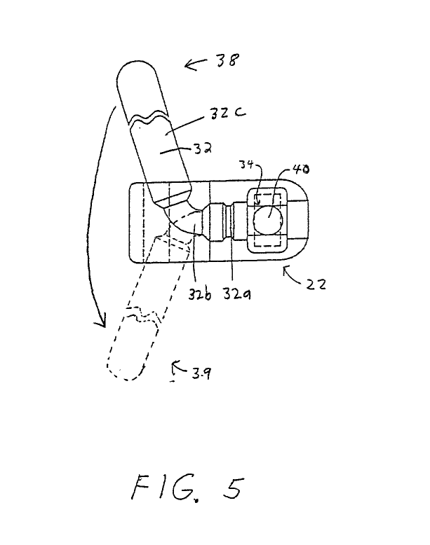

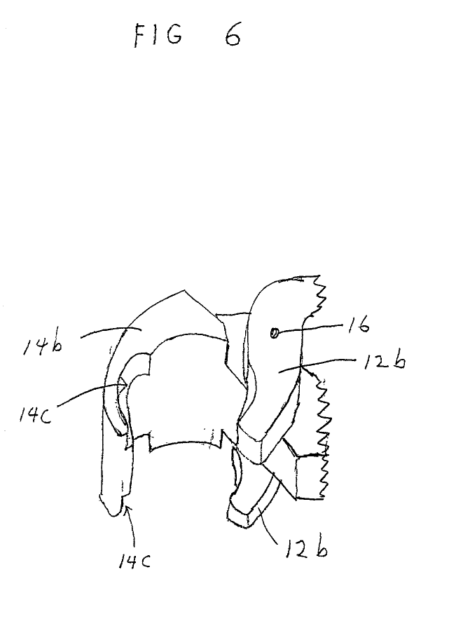

Referring to FIGS. 3 and 5, the rod 40 associates the scissors clamp 10, circle clamp 20 and the cam locking mechanism 30. The rod 40 has an eyehole 42 at one end through which the cam 34 may be inserted. At the opposite end, the rod 40 may be connected to a nut 70. A spring 80 surrounds the rod 40 between the nut 70 and the lower handle 14a of the scissors clamp 10. Alternatively, the rod 40 may be directly attached to the lower handle 14a of the scissors clamp 10.

Referring to FIGS. 3, 5, and 6, the universal scissors joint is engaged by rotating the cam handle 32 from an open position 38 to a locked position 39. Rotating the cam handle 32 rotates the cam 34 within the eyehole 42. This pushes the rod 40 upward, which causes the nut 70 and spring 80 to press upward on the lower handle 14a of the scissors clamp 10. Because the upper handle 12a of the scissors clamp 10 is connected by the bushing 50 to the lower portion 24 of the circle clamp 20, and the circle clamp 20 is a single piece, as the nut 70 and spring 80 move upward, both the scissors clamp 10 and the circle clamp 20 are squeezed, creating a tighter grip on the objects being held within the clamps.

Referring to FIGS. 1, 3, 5, and 6 the scissors clamp 10 and the circle clamp 20 are able to rotate with respect to each other. This allows any attached rods or surgical devices to be positioned in any manner desired for surgery. The ability to rotate may be locked or unlocked by the locking mechanism 30. When the cam handle 32 is in the open position 38, the scissors clamp 10 and the circle clamp 20 are able to freely rotate with respect to each other. When the cam handle 32 is in the locked position 39, the ability of the two clamps to rotate with respect to each other is made extremely difficult, with the result establishing a fixed position for the clamps with respect to each other so long as the cam handle 32 is in the locked position 39. As the cam handle 32 is rotated into the locked position 39, the upper handle 12a of the scissors clamp 10 is pressed against the bushing 50 with greater force, and the lower portion 24 of the circle clamp 20 is also pressed against the bushing 50 with greater force. This greater force creates greater friction between the scissors clamp 10 and the bushing 50 and between the circle clamp 20 and the bushing 50, greatly restricting the ability of the scissors clamp 10 and the circle clamp 20 to rotate with respect to each other.

Although the present invention has been described in considerable detail with reference to certain preferred versions thereof, other versions are possible. For example, types of clamps other than the circle clamp 20 may be used in conjunction with the scissors clamp 10, and more than two clamps may be used in one device. It will be appreciated that different sizes and shapes of the clamps may be used without departing from the scope of the present invention. Different types of cam locking mechanisms may be used, such as that revealed in U.S. Pat. No. 5,888,197. Still other types of locking mechanisms may be employed, such as a threaded locking mechanism. It will be appreciated that the handle and the cam may assume different shapes without departing from the scope of the present invention. It will be appreciated that the positions that constitute the locked and unlocked position may be changed without departing from the scope of the present invention. The revealed embodiment is not able to be completely disassembled, so as to allow sterilization without disassembly, but other embodiments may be completely disassembled. Therefore, the spirit and scope of the appended claims should not be limited to the description of the preferred versions contained herein.

What is claimed is:

1. A universal joint apparatus comprising:
a clamping member comprising a first segment and a second segment, the segments fastened by a pivot at an intermediate point of at least one segment;
a locking mechanism; and
a rod associating the locking mechanism with the clamping member.

2. The apparatus of claim 1, wherein the segments of the clamping member are arranged adjacent to each other on one side of the pivot, the segments crossing each other at the pivot so that:
the first segment comprises the upper portion of the first clamping member on a first side of the pivot and also comprises the lower portion of the first clamping member on the second side of the pivot; and
the second segment comprises the lower portion of the first clamping member on the first side of the pivot and also comprises the upper portion of the first clamping member on the second side of the pivot.

3. The apparatus of claim 1, further comprising at least one additional clamping member.

4. The apparatus of claim 3, wherein the at least one additional clamping member comprises an upper segment and a lower segment, said segments being parallel to each other;
having a space between them; and
being joined at one end.

5. The apparatus of claim 3, wherein the locking mechanism comprises a cam locking mechanism.

6. The apparatus of claim 5 wherein the cam locking mechanism comprises:
a handle; and
a cam connected to the handle and engaging the rod so that the cam operates the clamping members when selectively positioned by using the handle.

7. The apparatus of claim 6 wherein the rod comprises an eyehole.

8. The apparatus of claim 7 wherein the cam passes through the rod via the eyehole.

9. The apparatus of claim 7 wherein the cam comprises an axle that passes through the eyehole.

10. The apparatus of claim 3, further comprising at least one bushing placed between the clamping members.

11. The apparatus of claim 3, wherein the first clamping member and the additional clamping members are independently rotatable with respect to each other.

12. The apparatus of claim 11, wherein the locking mechanism, first clamping member, and the additional clamping members are independently rotatable with respect to each other.

13. The apparatus of claim 1, further comprising a nut attached to the end of the rod.

14. A universal joint apparatus comprising:
a first clamping member comprising a first segment and a second segment, the segments fastened by a pivot at an intermediate point of at least one segment;
a locking mechanism;
at least one other clamping member; and
a rod associating the locking mechanism, the first clamping member and the other clamping members.

15. The apparatus of claim 14, wherein the segments of the first clamping member are arranged adjacent to each other on one side of the pivot, the segments crossing each other at the pivot so that
the first segment comprises the upper portion of the first clamping member on a first side of the pivot and also comprises the lower portion of the first clamping member on the second side of the pivot;
and the second segment comprises the lower portion of the first clamping member on the first side of the pivot and also comprises the upper portion of the first clamping member on the second side of the pivot.

16. The apparatus of claim 14, wherein the at least one additional clamping member comprises an upper segment and a lower segment, said segments being parallel to each other;
having a space between them; and
being joined at one end.

17. The apparatus of claim 14 wherein the locking mechanism comprises a cam locking mechanism.

18. The apparatus of claim 17 wherein the cam locking mechanism comprises:
a handle; and
a cam connected to the handle and engaging the rod so that the cam operates the first clamping member and the additional clamping members when selectively positioned by using the handle.

19. The apparatus of claim 18 wherein the rod comprises an eyehole.

20. The apparatus of claim 19 wherein the cam passes through the rod via the eyehole.

21. The apparatus of claim 19 wherein the cam comprises an axle that passes through the eyehole.

22. The apparatus of claim 14, further comprising at least one bushing placed between the clamping members.

23. The apparatus of claim 14, wherein the clamping members are independently rotatable with respect to each other.

24. The apparatus of claim 23, wherein the locking mechanism, first clamping member, and additional clamping members are independently rotatable with respect to each other.

25. The apparatus of claim 14, further comprising a nut attached to the end of the rod.

26. A universal joint apparatus comprising:
a first clamping member comprising a first segment and a second segment, the segments fastened by a pivot at an intermediate point of at least one segment;
a locking mechanism;
a second clamping member; and
a rod associating the locking mechanism, the first clamping member and the second clamping member.

27. The apparatus of claim 26, wherein the segments of the first clamping member are arranged adjacent to each other on one side of the pivot, the segments crossing each other at the pivot so that
the first segment comprises the upper portion of the first clamping member on a first side of the pivot and also comprises the lower portion of the first clamping member on the second side of the pivot;
and the second segment comprises the lower portion of the first clamping member on the first side of the pivot and also comprises the upper portion of the first clamping member on the second side of the pivot.

28. The apparatus of claim 26, wherein the second clamping member comprises an upper segment and a lower segment, said segments being parallel to each other;
having a space between them; and
being joined at one end.

29. The apparatus of claim 26 wherein the locking mechanism comprises a cam locking mechanism.

30. The apparatus of claim 29 wherein the cam locking mechanism comprises:
a handle; and
a cam connected to the handle and engaging the rod so that the cam operates the first clamping member and the second member when selectively positioned by using the handle.

31. The apparatus of claim 30 wherein the rod comprises an eyehole.

32. The apparatus of claim 31 wherein the cam passes through the rod via the eyehole.

33. The apparatus of claim 31 wherein the cam comprises an axle that passes through the eyehole.

34. The apparatus of claim 26, further comprising a bushing placed between the first clamping member and the second clamping member.

35. The apparatus of claim 26, wherein the first clamping member and the second clamping member are independently rotatable with respect to each other.

36. The apparatus of claim 35, wherein the locking mechanism, first clamping member, and second clamping member are independently rotatable with respect to each other.

37. The apparatus of claim 26, further comprising a nut attached to the end of the rod.
